(19) United States
(12) Patent Application Publication
(10) Pub. No.: US 2012/0029348 A1
(43) Pub. Date: Feb. 2, 2012

(54) METHOD AND APPARATUS FOR MULTIMODAL IMAGING OF BIOLOGICAL TISSUE

(75) Inventors: Anna N. Yaroslavsky, N. Andover, MA (US); Elena Salomatina, Cambridge, MA (US); Richard R. Anderson, Boston, MA (US)

(73) Assignee: The Gemeral Hospital Corporation, Boston, MA (US)

(21) Appl. No.: 13/203,121

(22) PCT Filed: Apr. 13, 2010
(86) PCT No.: PCT/US10/30880

§ 371 Date: Oct. 13, 2011

Related U.S. Application Data
(60) Provisional application No. 61/169,258, filed on Apr. 14, 2009.

(51) Int. Cl. A61B 6/00

(52) U.S. Cl. 600/431; 600/476

(57) ABSTRACT

The present invention is directed to a novel multi-wavelength imaging method and apparatus that enables rapid imaging of tissue regions with accurate identification of tissue types within the region. Optical properties, such as co-polarized or cross-polarized fluorescence or reflectance intensity, optical density and/or reflectance, can be determined at a plurality of locations within the tissue region for each wavelength. Said properties at the two wavelengths, including calculated derivatives of the optical property with respect to wavelength, can be analyzed to image tissue structures and identify tissue types within the tissue region more accurately than can be achieved based on properties measured at a single wavelength.

### CROSS REFERENCE TO RELATED APPLICATION

The present application claims priority from U.S. Provisional Patent Application Ser. No. 61/169,258 filed Apr. 14, 2009, the disclosure of which is incorporated herein by reference in its entirety.

### FIELD OF THE INVENTION

The present invention is directed to a method and apparatus that facilitates accurate delineation of cancerous tissue or other particular types of biological tissue in tissue samples by comparing absorbance properties and/or other optical interactions of dyes by different tissue types at two or more particular wavelengths. The method and apparatus can be used to provide, among other things, real-time intraoperative delineation or demarcation of nonmelanoma skin cancers (e.g., basal cell carcinomas).

### BACKGROUND INFORMATION

Nonmelanoma skin cancers are a very common form of human cancer. Statistically, about one out of four Caucasians will develop at least one lesion during their life time. Basal cell carcinomas (BCCs) constitute about 80% of all nonmelanoma skin cancers, and their incidence has been increasing at a dramatic rate. They commonly appear on sun-exposed areas of the body such as the head and neck, and it may therefore be imperative to preserve normal skin surrounding these tumors. BCCs are disfiguring but rarely fatal (e.g., they lead to about 3000 deaths per year). However, due to their prevalence, the cost of their treatment exceeds $600 million per year.

BCCs are commonly treated by surgery. As these tumors often occur on the face and rarely metastasize, it is important to preserve as much healthy tissue as possible when excising them. However, in many cases the contrast of the lesions is poor and these tumors have poorly defined boundaries, which complicates visual tumor localization and precise excision.

Mohs micrographic surgery (MMS) is a clinical technique that allows complete control of excision margins during the operation. This technique uses detailed mapping and microscopic control of the excised lesion, which can pinpoint areas at the surgical margins involved with cancer that are otherwise invisible to the naked eye. While precise and accurate, MMS is a time-consuming and staff-intensive procedure that generally requires a surgeon trained in dermatopathology, a dedicated laboratory and a technician to prepare and evaluate frozen sections. Because of these shortcomings, MMS is used in a minority of cases. Thus it is imperative that new techniques and treatment protocols be set in place to address the problem of intraoperative margin control.

Recent advances in optical imaging have made accurate, real-time and cost-effective intraoperative inspection of the entire BCC tumor margin technically feasible. However, these techniques generally lack one or more elements necessary for their practical use in a clinical setting. FDA-approved contrast agents or dyes suitable for in vivo use, such as methylene blue (MB), are often applied to the lesions, as the intrinsic differences in optical signals from normal and diseased tissues are often subtle. MB has been successfully applied to grossly demarcate neoplastic tumors in the bladder, tumors of the pancreas, and Barrett's esophagus metaplasia. Even though the MB dye tends to accumulate to a much greater extent in the mitochondria of cancerous cells as compared to normal cells, it is not 100% specific. Healthy skin tissue structures, such as hair follicles, sebaceous glands and epidermis, can also retain some dye. However, the appearance of these tissues and the concentration of the dye differ considerably from those of cancer.

Several complementary techniques, including dye-enhanced reflectance and fluorescence polarization imaging (RFPI), are capable of providing real-time high contrast images of skin cancer with a success rate of up to 94%. Such techniques are described, e.g., in U.S. Pat. No. 7,289,205. However, such techniques may not be able to reliably distinguish between cancerous tissue and certain tissue structures, e.g., hair follicles or the like, that may exhibit similar optical densities.

Thus, in view of the above-described deficiencies, a simpler, more accurate, and/or time-efficient method would be desirable for mapping tumor borders and for improved distinguishing between tumor tissue and healthy tissue.

### SUMMARY OF THE INVENTION

The present invention is directed to meeting the aforementioned needs and addressing the deficiencies particularly discussed above and generally in the prior art. Embodiments of the present invention provide a method and apparatus that includes analysis of certain wavelength-dependent optical properties and, optionally, corresponding wavelength derivatives of such optical properties, at a plurality of locations within tissue samples that include both healthy tissue structures and other tissue structures (e.g., cancerous tissue such as BCCs). The optical properties can include local reflectance or optical density of tissue that has been treated with a contrast agent, e.g. a dye such as methylene blue (MB). Polarization information can also be used at each wavelength. Such method and apparatus can facilitate delineation of nonmelanoma skin cancer regions, for example, when used in conjunction with a dye-enhanced reflectance and/or fluorescence polarization imaging procedure.

In one embodiment of the present invention, a tumor demarcation technique can be provided that includes imaging a tissue region or sample treated with a contrast agent such as a dye at two or more particular wavelengths. Imaging light can be provided by monochromatic light sources, or by a broadband light source and two or more monochromatic filters, which can optionally be polarizing filters. A local optical density, reflectance, or other optical property associated with different tissue structures, e.g., various healthy skin structures and cancerous tissue, may exhibit a different variation with respect to wavelength for different structures, even when the values of such properties may be similar for certain tissue types or structures. Accordingly, a method and apparatus for image analysis of tissue using this technique can provide a quick spectroscopic analysis at as few as two (2) interrogation wavelengths that may be sufficient to more reliably distinguish a structure such as cancerous tissue from surrounding healthy tissue. Embodiments of the present invention may therefore provide a method and apparatus that facilitates real-time accurate intraoperative identification and delineation of cancerous tissues or other tissue structures.

In another embodiment of the invention, wavelength-dependent derivatives of such optical properties can be determined and used to image a tissue region and/or distinguish different tissue types within the region. Such derivatives are preferably estimated based on measurements taken at wavelengths that differ by at least 5 nm, e.g., wavelengths that differ by between about 5 nm and 30 nm, or between about 10 nm and 20 nm.

In a further embodiment of the invention, the contrast agent can be methylene blue when in vivo tissue imaging is performed. At least one of the wavelengths used with this contrast agent is preferably between about 600 nm and about 735 nm. For example, a wavelength of about 615 nm and/or about 665 nm may be used, as these wavelengths correspond to absorption maxima of methylene blue and thereby can provide large signals that facilitate identification and/or delineation of different tissue types. When imaging tissue ex vivo, a contrast agent such as toluidine blue can be used.

In still further embodiments of the invention, other wavelengths can be used based on the tissue being examined. For example, a wavelength close to about 410 nm can be used if the tissues to be imaged or distinguished have significantly different levels of blood content. A wavelength between about 1100 nm and about 1300 nm may also be used if imaging based primarily on scattering behavior is desirable.

In a yet further embodiment of the invention, fluorescence polarization and/or reflectance polarization information can be obtained at a plurality of locations within the tissue region at the two or more wavelengths. Such information can also be used to image and/or delineate different tissue structures and types within the tissue region.

In yet another embodiment of the invention, the optical properties determined at two or more wavelengths within the tissue region can be compared to predetermined wavelength-dependent values of such properties for various tissue types. Such comparison of local optical properties at more than one wavelength can provide more accurate identification and delineation of tissue types as compared with measurement of optical properties obtained at a single wavelength. Such predetermined optical properties used for comparison with detected or measured values in a tissue sample can also include derivatives of the measured optical properties with respect to wavelength.

In another embodiment of the invention, an apparatus is provided that includes a light source configured to irradiate a tissue sample with light having at least two distinct wavelengths, one or more detectors configured to detect remitted light form the tissue sample at each wavelength, and an analyzer configured to image, identify and/or delineate different tissue types or structures within the tissue sample as described herein. The apparatus can optionally include polarizing filters if polarization properties of the tissue are used to image the tissue sample.

The methods, system and apparatus provided in accordance with the present invention may be particularly useful for real-time intra-operative imaging and delineation of nonmelanoma skin tumor margins, accelerating the tumor excision process and increasing the number of surgical procedures that can be performed. For example, embodiments of the present invention can be used to guide an actual tumor excision surgery. Such improvements can be provided by incorporating spectral analysis into RFPI procedures and systems that facilitate real-time tumor delineation processes.

These and other objects, features and advantages of the present invention will become more apparent from the following detailed description taken in conjunction with the drawings.

### BRIEF DESCRIPTION OF THE DRAWINGS

So that those having ordinary skill in the art to which the present application appertains will more readily understand how to make and use the same, reference may be had to the drawings wherein:

FIG. 1 is a schematic illustration of an exemplary apparatus configured to perform reflectance and fluorescence polarization imaging;

FIG. 2a is an exemplary grayscale superficial reflectance image at 665 nm image of a 16×7 mm scalp tissue specimen containing a basal cell carcinoma;

FIG. 2b is a fluorescence polarization image excited at 615 nm and registered in the range between 660 nm and 750 nm of the tissue specimen shown in FIG. 2a;

FIG. 2c is an image of an H&E frozen histology of the tissue specimen shown in FIG. 2a with the tumor borders outlined by a certified pathologist;

FIG. 2d is a graph of averaged optical density spectra obtained for cancerous tissue and healthy tissue structures;

FIGS. 3a-3e are plots of optical density spectra for BCC—healthy tissue structure pairs, averaged over 20 samples: (a) BCC and sebaceous gland, (b) BCC and hair follicle, (c) BCC and epidermis, (d) BCC and collagen, and (e) BCC and subcutaneous fat; and

FIGS. 4a-4e are plots of wavelength-dependent optical density derivatives, averaged over 20 samples, for BCC—healthy tissue structure pairs: (a) BCC and sebaceous gland, (b) BCC and hair follicle, (c) BCC and epidermis, (d) BCC and collagen, and (e) BCC and subcutaneous fat.

While the present invention will now be described in detail with reference to the figures, it is done so in connection with the illustrative embodiments and is not limited by the particular embodiments illustrated in the figures.

### DETAILED DESCRIPTION

Biological contrast agents, e.g. stains or dyes such as MB, can distribute differently in different types of tissue. Such variations in dye distribution can lead to differences in spectral absorption and other optical properties, which can depend in part on differences in local dye concentrations. Other factors may also affect the observed optical properties, such as local concentration of blood/hemoglobin or other light-absorbing substances in the tissues. In conventional tissue identification techniques, optical properties of dyed tissue measured using a broadband source or a single wavelength can be compared to identify differences in tissue types based on variation in the dye distribution. However, certain distinct tissue types may exhibit similar optical properties in such measurements, which can impair the identification or differentiation of tissue types in a sample.

Certain optical properties at two or more particular wavelengths can be measured at a plurality of locations within a region of a tissue sample. Differences in the properties at each wavelength, together with differences in such properties at the different locations within the region, can be compared or analyzed and used to provide images of the tissue region and more accurate and/or reliable identification of tissue types within the sample.

The measured optical properties can include, for example, reflectance of regions of a tissue sample (e.g., absolute reflectance spectra), optical density, etc., or combinations thereof. A particular optical property may be selected based on the tissue being examined. For example, the reflectance can be measured directly, but the reflectance value generally depends in part on the thickness of the sample. In contrast, the optical density is an intrinsic property of the tissue, but its determination is based on certain assumptions about the tissue itself. Various polarizations of light, non-polarized light, or combinations thereof may also be used to facilitate delineation of tissue types as described herein. The types of images used for optical analysis can be based on, for example, non-polarized light, or various combinations of co-polarized intensities (Ico) and cross-polarized intensities (Icross). Such combinations can include spectral data based on signal differences (e.g., Ico-Icross), ratios (e.g., Ico/Icross), relative differences (e.g., (Ico-Icross)/(Ico+Icross)), etc. In typical applications, delineation of deeper tissues may be improved when using signals based on cross-polarized light, whereas more superficial tissues may be better delineated using differences between co-polarized and cross-polarized intensities.

Various tissue dyes or stains may be used. Methylene blue (MB) is an FDA-approved stain that may be used in vivo as well as in ex vivo tissue samples. Other dyes may also be used, particularly for ex vivo tissue analysis. For example, toluidine blue (TB) may be used to stain ex vivo tissue samples for analysis as described herein. TB can shift the absorption spectrum towards the yellow region of the spectrum as compared to MB, which is generally closer to the absorption region of blood. Accordingly, TB may be preferable for analysis of tissues that do not contain a lot of blood.

The optical properties of certain regions of a tissue sample can be measured as described herein at two or more wavelengths. One of the wavelengths used can be at or close to a local maximum or peak in the dye absorption spectrum. For example, if MB dye is used, one of the wavelengths can be at or close to 615 nm or 665 nm, which correspond approximately to local absorption maxima for MB. In general, wavelengths in the vicinity of such maxima may be preferable (e.g., between about 600 nm and about 685-700 nm for MB) because such wavelengths can provide larger signals and corresponding large differences between signals, facilitating differentiation. If another dye is used, different wavelengths at or close to absorption peaks for the particular dye may be used. Using at least one wavelength that is near an absorption maximum can provide a larger signal for the measured intensity, which can facilitate differentiation between such signals for different tissue types and regions in the sample.

Optical properties (e.g., absorption intensities) can also be obtained at or near these wavelengths. Accordingly, the relative absorption behavior at two or more wavelengths can be compared for the different tissue regions, rather than just comparing an absolute intensity at a single wavelength (or a single distribution of wavelengths from a broadband source). Such analysis of relative optical interactions of tissues with a dye at different wavelengths can provide a more detailed and/or accurate differentiation between tissue types in the different tissue regions of a sample.

Analysis of derivatives of wavelength-dependent optical properties as described herein can also provide or improve delineation of tissue types in regions of a sample. For example, derivatives in values of certain optical properties with respect to wavelength can be estimated at a particular wavelength. Such property derivatives can provide differentiation between tissue types or regions even if absolute values of such optical properties are the same or similar for different tissue types.

The wavelength derivative information for optical properties can be calculated using conventional mathematical techniques. For example, a particular optical property such as those described herein can be determined at a particular location in the tissue sample at two different wavelengths. The difference between wavelengths used to estimate derivative information is preferably greater than about 5 nm, for example, between about 5 nm and about 30 nm, between about 5 nm and about 20 nm or between about 5 nm and about 15 nm, e.g., about 10 nm. The difference in wavelengths used to calculate the derivative information as described herein is preferably large enough to generate distinguishable values of the optical property measured at the different wavelengths, and small enough to provide an accurate estimate of the local derivative(s) of the property with respect to wavelength.

A first derivative can be estimated using a conventional linear formula, e.g., by taking the difference between the values of the measured property at each wavelength, and dividing this difference by the difference in the wavelengths at which the property was measured. For example, the derivative at a wavelength b can be estimated by measuring the optical property P at two wavelengths b and c, where c=b+δ. Preferably, δ is at least about 5 nm, for example, between about 5 nm and about 30 nm, between about 5 nm and about 20 nm or between about 5 nm and about 15 nm, e.g., about 10 nm. The approximate value of the derivative of an optical property P measured at wavelength b, which can be expressed as dP(b)/dλ (where λ represents wavelength), can be expressed as [P(c)-P(b)]/(c-b). This procedure provides an estimate of the first derivative of the optical property value with respect to wavelength at wavelength b+δ/2, which can be close to b if δ is small.

In a further embodiment, second-derivative information of the optical property with respect to wavelength can also be used to image tissue and/or identify particular tissue structures. The second derivative of an optical property with respect to wavelength can be estimated using a conventional 3-point technique. The derivative at a wavelength b can be estimated by measuring the optical property P at three wavelengths a, b, and c, where a=b−δ and c=b+δ. Preferably, δ is at least about 5 nm, for example, between about 5 nm and about 30 nm, between about 5 nm and about 20 nm or between about 5 nm and about 15 nm, e.g., about 10 nm. The second derivative of P at wavelength b, which can be written as d²P(b)/dλ², can be approximated as [P(a)−2*P(b)+P(c)]/δ². This expression provides an estimate of the second derivative of the optical property value with respect to wavelength at wavelength b.

Derivative information used to image and/or identify tissue structures within a tissue sample can include first-derivative information, second-derivative information, or both. The derivative information can be calculated from optical property measurements made at particular locations in the sample at different wavelengths as described herein above.

A database or table of such derivative information can also be prepared for particular tissue structures or tissue types (e.g., hair follicle, tumor, sebaceous gland, etc.) by first measuring the optical property of a sample of the tissue type at a plurality of wavelengths, and then calculating the derivative information as described above. Such derivative information can be stored in a computer-readable medium, and can then be compared to such derivative information obtained at a particular location within the tissue sample to facilitate accurate identification and/or imaging of the tissue type within the sample. Such derivative information can include, e.g., a first derivative and/or a second derivative of the optical property with respect to a wavelength at which the property is measured. Further details of such a procedure are described herein below.

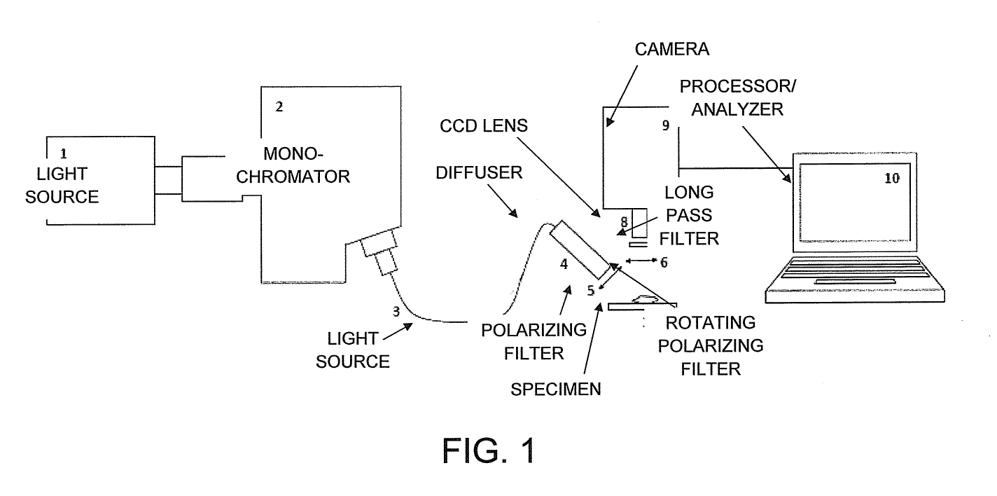

A schematic illustration of an exemplary system that can be used for polarization-enhanced reflectance and fluorescence imaging is presented in FIG. 1. A 500 W Xe-Arc lamp 1 (Spectra Physics Oriel, Stratford, Conn.) coupled with an MS 275 grating monochromator 2 (Oriel Instruments, Stratford, Conn.) was used as a light source. The light from this source 1 was delivered to a tissue specimen 11 placed on a sample stage via the light guide 3 (Oriel Instruments, Stratford, Conn.). A holographic diffuser 4 (15⁰, Edmund Optics, Barrington, N.J.) and a linearly polarizing filter 5 (Meadowlark Optics, Frederick, Colo.) were introduced into the path of the incident light to provide a homogeneous linearly polarized illumination of the specimen.

A CCD CoolSnap HQ camera 9 (Roper Scientific, Tucson, Ariz.), equipped with an achromatic CCD lens 8 (Linos Photonics Inc., Milford, Mass.) and a rotating linearly polarizing filter 6 (Meadowlark Optics, Frederick, Colo.), was used to acquire co- and cross-polarized reflectance images of the specimens. For fluorescence imaging, an additional long pass filter 7 (660AELP, Omega Optical, Brattleboro, Vt.) was attached to the camera lens. The CCD camera 9 was provided in communication with a computer 10 or other processing arrangement that can be configured to store, process and/or display the image data, as well as to perform spectrographic analysis as described herein.

The angle between the excitation and emission polarizer was approximately 55⁰. The maximal field of view provided by the system was 3.3×2.9 cm, and observed lateral resolution was better than 25 μm. Axial resolution varied between about 70 to 200 μm. The power density of the light on the tissue did not exceed 0.3 mW/cm². Illumination, acquisition, and image processing were controlled via codes developed using the Metamorph 6.0rl imaging software (Molecular Devices Corporation, CA). The reflectance measurements were obtained in approximately 3 seconds or less for both polarizations, and the fluorescence imaging was obtained in about 5 seconds or less.

The apparatus shown in FIG. 1 can be used to obtain high-contrast reflectance and fluorescence polarization images of skin cancer or the like as described, e.g., in U.S. Pat. No. 7,289,205. The monochromator 2 can further be configured to provide light from the Xe-Arc lamp 1 at particular wavelengths onto a tissue specimen 11. Reflected light at these particular wavelengths can be detected by the CCD camera 9, and signals from the CCD camera 9 can be provided to the computer 10. The computer 10 can be configured to analyze the intensities of light reflected from certain regions of the specimen 11 at particular wavelengths, using exemplary data and procedures described herein, to better distinguish, e.g., healthy tissue from cancerous tissue in these regions of the tissue sample 11.

Twenty (20) freshly-excised thick skin specimens with residual nonmelanoma skin cancers were obtained from 20 patients under an IRB-approved protocol from Mohs micrographic surgeries performed at the Dermatologic Surgery Unit of Massachusetts General Hospital. Characteristics of the lesions are summarized in Table 1.

Commercially available MB dye (methylene blue injection, 1%, American Regent Inc., Shirley, N.Y.) was used in the present technique. The MB dye was diluted to a concentration of 0.2 mg/ml using Dulbecco's phosphate-buffered solution (DPBS 1X, pH 7.4, Mediatech, Herndon, Va.) and kept at a temperature of 37° C. The specimens were soaked in the diluted MB dye for up to 2 minutes. To remove an excess of the dye, the specimens were briefly rinsed in saline solution having a pH of 7.4. Each tissue specimen was then placed, dermal side up, into a Petri dish on gauze moistened with saline solution, covered with a microscopic slide, and imaged. All imaging procedures of the tissue specimens were performed at room temperature.

Superficial reflectance images were calculated as a difference of experimentally acquired co- and cross-polarized images in the range of 395 nm to 735 nm with steps of 10 nm. Fluorescence polarization images of the tissues were calculated as:

F=(F_{co}-F_{cross})*100/(F_{co}+F_{cross}), (1)

where F is a fluorescence polarization image, and F_{co} and F_{cross} are co- and cross-polarized fluorescence emission images, respectively. Image processing was performed in real time.

Co-polarized reflectance images were obtained and used to determine the values of diffuse reflectance and the optical density for each skin structure in the image at a particular wavelength. The spectral responses, e.g., optical densities and corresponding wavelength derivatives, of the following structures were analyzed and stored in a database: cancerous tumor, hair follicles, sebaceous glands, epidermis, collagen, and subcutaneous fat.

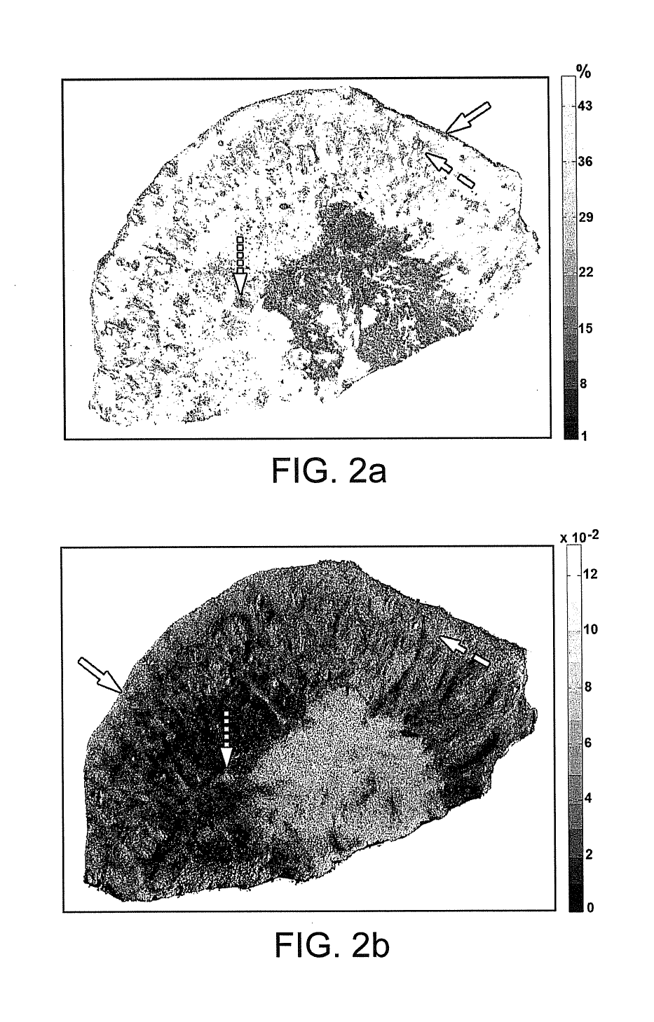

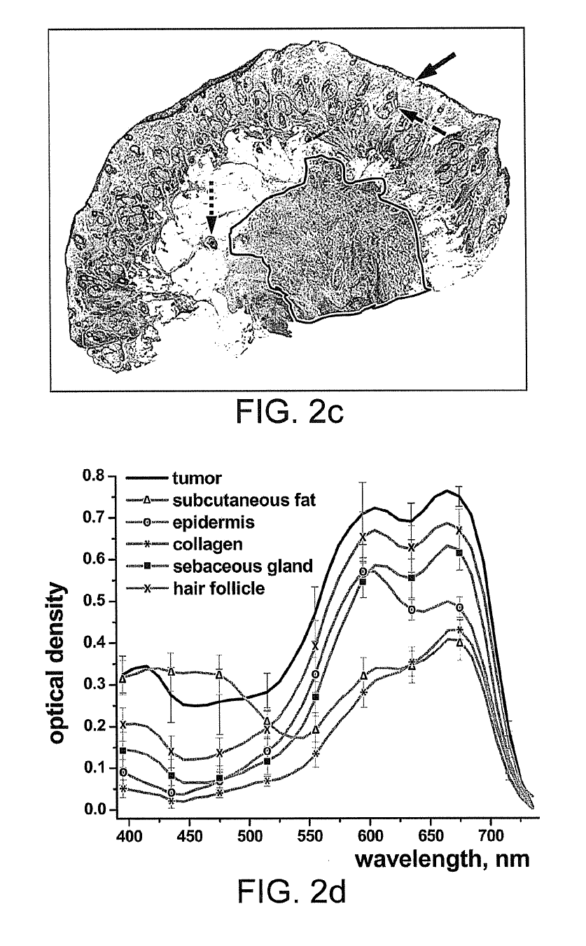

Stacks of 35 co-polarized reflectance light images were acquired in the wavelength range from 395 nm to 735 nm, at intervals of 10 nm. A calibrated gray reference (having a reflectance value of about 35% in the range from 395 nm to 735 nm) was placed in the camera field of view to facilitate absolute quantification of diffuse reflectance. Each experimental spectrum was divided by the value of the diffuse reflectance measured at 735 nm. Because this wavelength lies outside the absorption band of the MB dye, this normalization provides a compensation for possible effects of background absorption and scattering on the spectral analysis. Exemplary grayscale-coded quantitative reflectance and fluorescence polarization images of a BCC are provided in FIGS. 2a and 2b, respectively.

For each co-polarized reflectance image acquired at each wavelength, the absolute diffuse reflectance and optical density of the skin structures were calculated using the following expressions:

R^{λ}=0.35*R^{λ}ₛ/R^{λ}_{ref}, (2)

and

OD^{λ}=log(1/R^{λ}), (3)

where R^{λ} represents an absolute diffuse reflectance of the skin structures, R^{λ}_{ref} represents a relative diffuse reflectance of the gray reference, R^{λ}ₛ represents a relative diffuse reflectance of the skin structures, and OD^{λ} represents an optical density of the skin structures, each at a particular wavelength. Wavelength derivatives of the observed optical densities were then calculated.

A Student's two-tailed t-test (Statistica 6.0, StatSoft, Inc, Tulsa, Okla.) was used to evaluate the significance of the difference in obtained optical densities and their wavelength derivatives between normal skin structures and cancerous tissues for a set of selected wavelengths. The differences in optical densities and corresponding wavelength derivatives between healthy and cancerous tissues were considered to be statistically significant when the calculated probability value (p-value) was equal to or less than 0.05. The results of this analysis were summarized in a table to facilitate identification of the spectral regions where the optical densities and their wavelength derivatives differed significantly between normal and cancerous tissues.

En face H&E stained frozen sections were processed from the imaged tissue blocks using standard Mohs micrographic surgery procedures. High-resolution images of the sections were obtained using a SPOT RT microscope digital camera (Diagnostic Instruments, Inc, Sterling Heights, Mich.) coupled with an Axiophot microscope (Carl Zeiss, Thornwood, N.Y.). The tumor margins in the H&E sections were determined by a certified pathologist who had no access to the optical images. The quantitative reflectance (at 665 nm) and fluorescence polarization light images were then compared to the marked histology.

An exemplary H&E histopathology, shown in FIG. 2c, can be compared to the reflectance and fluorescence polarization images shown in FIGS. 2a and 2b, respectively. The tumor in FIG. 2c is outlined by a certified pathologist. The location, dimensions, and shape of the tumor correlate well with the tumor margins identified in the histopathology of FIG. 2c. The BCC shown in the optical images of FIGS. 2a and 2b exhibits a higher contrast as compared with the histopathology generated at the same magnification in FIG. 2c.

The superficial reflectance image shown in FIG. 2a indicates that the highest optical density, which is coded as dark gray, corresponds to the location of the tumor. However, some parts of the epidermis (indicated by solid white arrows in the optical images of FIGS. 2a and 2b, and by solid black arrows in the histological image of FIG. 2c), hair follicles (indicated by dotted white arrows in FIGS. 2a and 2b, and by dotted black arrows in FIG. 2c), and sebaceous glands (indicated by dashed white arrows in FIGS. 2a and 2b, and by dashed black arrows in FIG. 2c), accumulate a considerable amount of dye and may be confused with the tumor.

Direct qualitative comparison of the optical images (FIGS. 2a and 2b) with the corresponding histopathology (FIG. 2c) suggests that epidermis, hair follicles and sebaceous glands may be discriminated from a tumor by their morphological appearances. However, these tissue structures may be involved with a tumor, and therefore a time-efficient method that could reliably detect small cancer nests and is compatible with reflectance polarization imaging would be valuable. In the fluorescence polarization image of FIG. 2b, the tumor exhibits a higher fluorescence polarization (shown as a lighter shade of gray) as compared to the normal tissues of the sample. Skin appendices, such as hair follicles and sebaceous glands, cannot be easily distinguished in this image. However, even in fluorescence polarization images there may be several bright pixels along the epidermis (indicated by a solid white arrow in FIG. 2b, which correspond approximately to dark pixels at the same location in FIG. 2a) and in the vicinity of the hair follicles (indicated by a dotted white arrow) that may manifest malignancy. Although the bright pixels along the epidermis may not be easily distinguishable against the white background in FIG. 2b, such features can be more readily discerned in a color-coded image that can be generated using the same image data.

Thus, several common skin tissue structures, such as hair follicles, sebaceous glands and epidermis, can retain some dye that may be applied for optical imaging of the tissue. Some of these structures can appear red in color-coded reflectance images and green in color-coded fluorescence images. To resolve ambiguities in characterizing such tissue structures, spectral responses of these skin structures present in the reflectance images were characterized.

FIG. 2d shows a wavelength dependency of the optical densities of MB dye-stained skin tissue containing a tumor and other structures, where such densities were averaged over the sample. The curves in FIG. 2d correspond to the following structures, in order from top to bottom at the rightmost peak (e.g., at about 680 nm): tumor, hair follicle, sebaceous gland, epidermis, collagen, and subcutaneous fat.

The MB dye concentration is the highest in the tumor, which results in the increase of optical density in the spectral range that corresponds to the two absorption maxima of methylene blue around 615 nm and 665 nm. Even though the concentration of the dye appears to be somewhat lower in hair follicles, the spectral responses of these two structures (tumor and hair follicles) are quite similar. The spectral responses of the sebaceous glands suggest that the optical density of these skin appendices is much lower in the MB dye absorption wavelength range between 570 nm and 710 nm, as compared to a cancer tumor.

In the optical density spectrum of the epidermis, the MB dye absorption peak at about 615 nm appears more pronounced than the second absorption peak at about 665 nm. This feature can facilitate a delineation between cancerous tumor tissue and epidermis, as the former exhibits an opposite relative magnitude of the two absorption peak intensities. In addition, the comparison of the optical density curves of epidermis and tumor shows that the tumor accumulates much more dye and exhibits a considerably higher optical density in the vicinity of the 665 nm MB absorption band.

Collagen and fat do not appear to accumulate significant amounts of the dye, and therefore can be more easily distinguished from the tumor. Further, subcutaneous fat spectra exhibit higher optical density in the wavelength range from 395 nm to 560 nm, as compared to the other skin structures. This observation may relate to a higher hemoglobin content in subcutaneous fat.

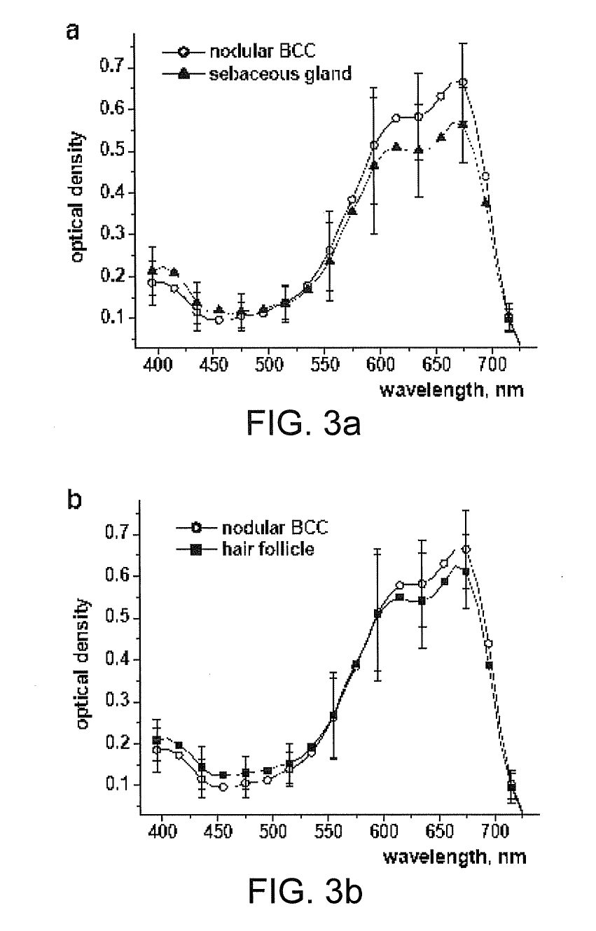

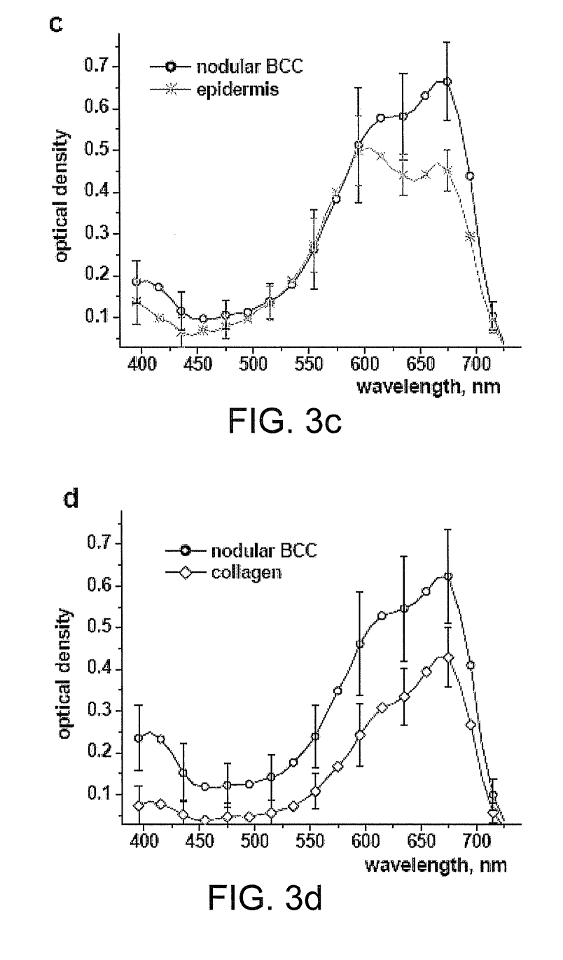

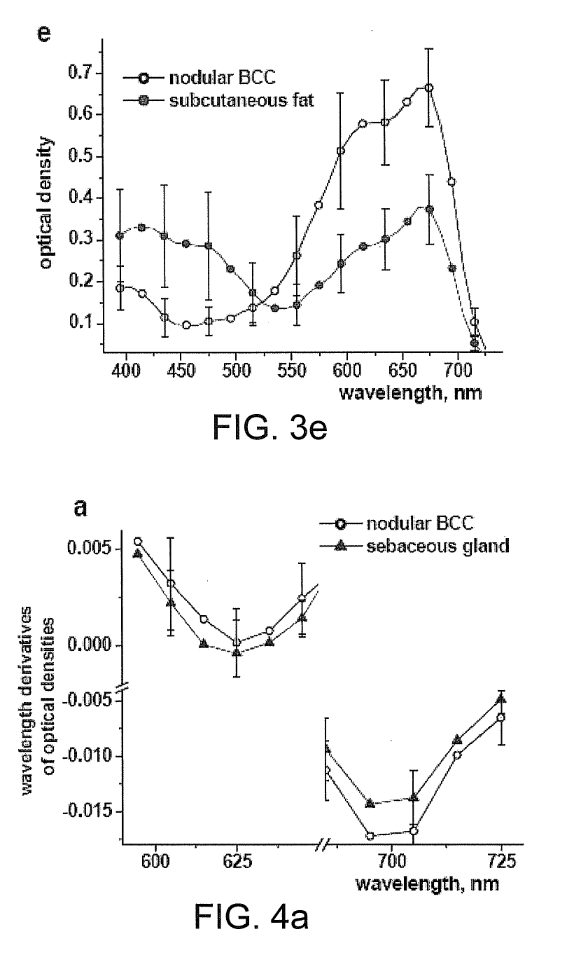

Thus, as shown in FIG. 2d, each of the studied skin structures exhibits a different spectral response within the absorption band of the MB dye, based in part on the different uptake of the dye by the various skin structures. To confirm that the spectral signatures of MB dye-stained skin structures can be used reliably for distinguishing cancer from benign tissue, the optical densities of cancer, hair follicles, sebaceous glands, epidermis, collagen and fat collected from 20 fresh thick samples containing BCCs were determined and statistically analyzed. The averaged dependences of the optical densities on the wavelength for all of the studied skin structures are shown in FIGS. 3a-3e.

The data shown in FIGS. 3a-3e indicate that the optical density of cancerous tissue appeared to be substantially higher than that of other skin tissues within the absorption bands of the MB dye. Additionally, the differences in the optical densities of epidermis and cancer were observed to be considerable for wavelengths between 390 nm and 485 nm. (See, e.g., FIG. 3c.) This spectral range corresponds approximately to the Soret absorption band of hemoglobin. For example, it has been observed that cancerous tissue generally contains blood, whereas healthy epidermis is generally bloodless. Therefore, the observed differences in optical density may be based in part on the difference in blood content of the tumor and the epidermis.

Due to variations in hemoglobin content and a relatively low dye uptake by collagen and fat, the differences in the optical density of collagen-tumor and fat-tumor pairs were significant over substantially the entire spectral range investigated, as shown in FIGS. 3d and 3e.

Differences in blood content between cancer and adjacent normal skin tissue may be highly variable. Further, reflectance measurements may be affected by oxygenation of blood as well as by location of the lesion on the body. Thus, identified differences in the spectral range of hemoglobin absorption cannot provide a generally reliable basis for tissue discrimination. The spectral analysis of tissue structure optical density presented herein indicates that for the various tissue structures investigated, with the possible exception of hair follicles (see FIG. 3b), the differences in optical density between a tumor and the healthy skin structures for wavelengths between 615 nm and 700 nm were significant. (See FIGS. 3a, 3c, 3d, and 3e.) Therefore, further analysis of spectral variations for optical image analysis may be focused on the spectral range between 600 nm and 735 nm.

To address the challenge of achieving reliable spectroscopic discrimination between a tumor and hair follicles, differences in the wavelength derivatives of the optical densities have been calculated and analyzed for cancer and normal tissue. For example, it has been observed that methylene blue dye can display different absorptive properties depending on the local biochemical environment. Thus, subtle observed deviations in the slopes of the MB-stained cancer and hair follicle optical density curves may be caused by differences in the biochemical environment of these two structures. Such deviations, if reproducible, may be used to facilitate a reliable differentiation of cancer from surrounding healthy tissue.

The derivatives of the optical properties of different tissues with respect to wavelength can be calculated based on measurements taken at wavelengths that are preferably greater than 5 nm apart. In general, there may be no significant or easily-resolvable changes in the optical properties of a tissue at two wavelengths that differ by less than 5 nm. For example, derivative information for an optical property of a tissue can be obtained by measuring the optical property at two or more wavelengths that differ by at least about 5 nm, for example, between about 5 nm and about 30 nm, between about 5 nm and about 20 nm or between about 5 nm and about 15 nm, e.g., about 10 nm. The wavelengths used to determine such derivative information preferably differ by less than about 30 nm to avoid traversing of local maxima or minima in the wavelength-dependent optical property when calculating such derivatives. The exemplary wavelength difference of about 10 nm used in the example described herein for calculating such derivatives can provide a good compromise between resolving optical property changes and accurate approximation of local wavelength-dependent derivatives.

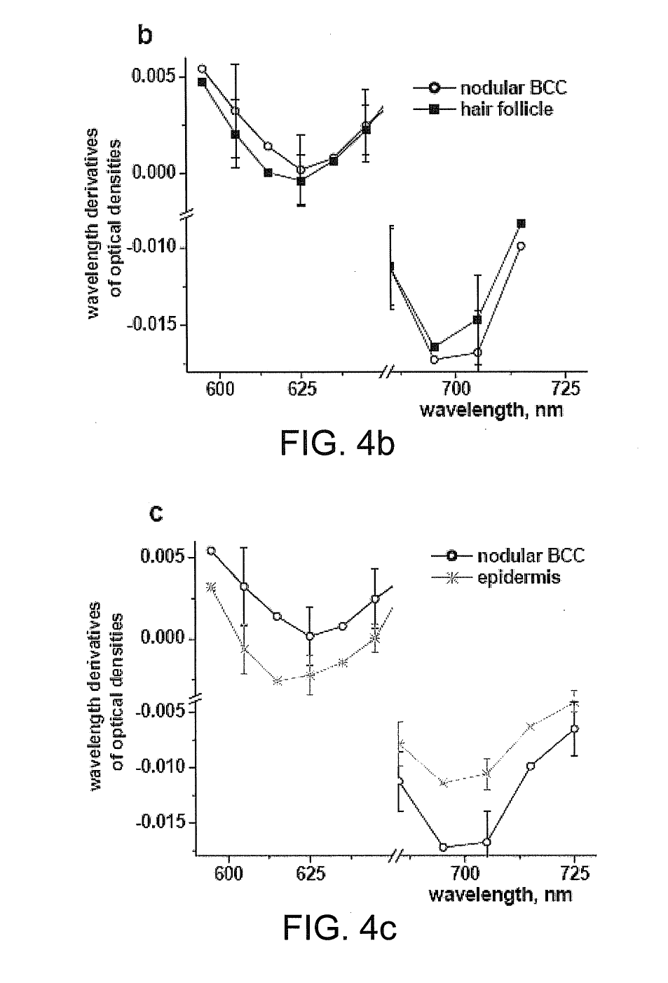

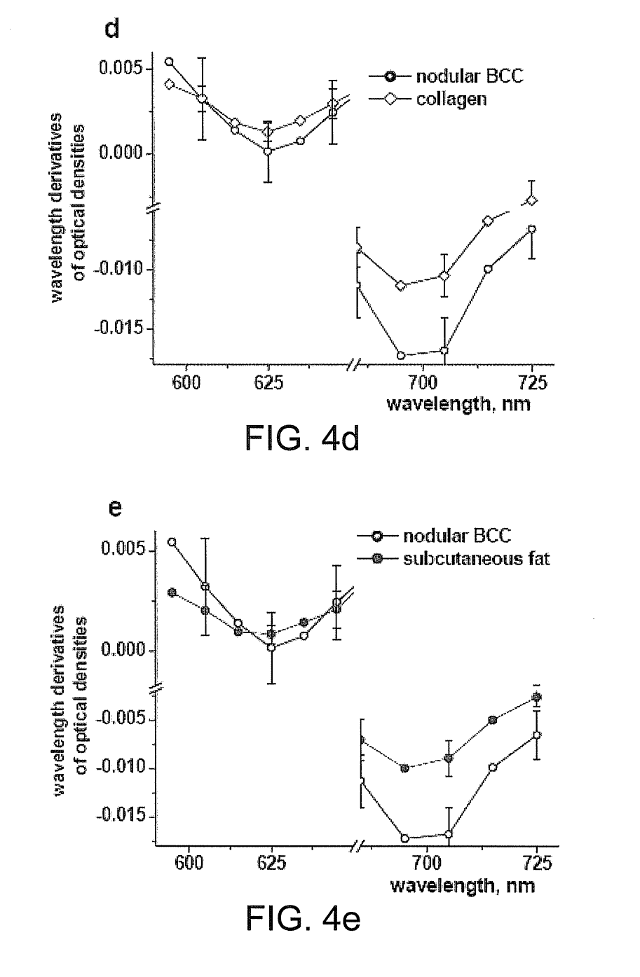

The resulting dependences of the wavelength derivatives of the optical densities for the various healthy tissue structures as compared with tumor tissue are presented in FIGS. 4a-4e. Unpaired two-tailed t-tests were used to evaluate the significance of the differences between optical density and the corresponding wavelength derivatives of normal (healthy) tissue structure and cancer tissue pairs. Pair-wise t-tests were utilized rather than starting with a standard ANOVA analysis to better identify regions of difference between the cancer tissue and other normal skin type tissues. Accordingly, independent healthy skin type tissue samples were individually tested against the cancer tissue samples.

The results of the comparative tests are summarized in Table 2. These data indicate that for all cancer-healthy tissue pairs, with the exception of hair follicles (shown in FIG. 4b), there are statistically significant differences in the optical density at a wavelength of 665 nm, which corresponds to one of the absorption maxima of MB dye. The largest statistically significant differences in the wavelength derivatives of the optical density between cancer and hair follicles were observed at wavelengths of 615 nm, 705 nm, and 735 nm, where 615 nm corresponds approximately to another local absorption maximum of the MB dye. (See FIG. 4b.)

Differences in the wavelength derivatives of the optical density were observed to be significant at 705 nm for all of the cancer-normal tissue pairs examined. Thus, particular spectral ranges suitable for reliable differentiation of cancerous and healthy skin tissue have been identified using spectral analysis of the reflectance images. These wavelength ranges include the 615 nm and 665 nm wavelengths, which correspond approximately to local absorption maxima of MB dye.

Accordingly, spectral analysis of the tissue images can be incorporated into the tumor detection procedure without significantly altering the image acquisition procedure or increasing the complexity and time needed to perform the procedure. For example, the exemplary apparatus shown in FIG. 1 can be adapted to perform the image acquisition and analysis procedures described herein. The monochromator 2 can be configured to provide light from the Xe-Arc lamp 1 at one or more particular wavelengths described herein where significant differences in the wavelength derivatives of the optical density between cancerous and healthy tissue were discovered. The computer 10 can be configured to analyze the intensities of this light reflected from certain regions of the specimen 11 and detected by the CCD camera 9. at these wavelengths. Comparing the optical density of the regions of the images at these wavelengths with the data provided herein can facilitate a more accurate distinguishing between healthy tissue and cancerous tissue in these regions of the tissue sample 11.

Although a complete spectroscopic analysis of every pixel in the image for the entire visible spectral range can be performed, this comprehensive approach may not be an optimal or even preferable approach for performing the tissue identification analysis described herein. For example, analysis of even ten (10) wavelengths for an image containing 1100×1300 pixels can require a prohibitively long time, rendering this approach unsuitable for intraoperative use. Further, such a detailed approach may be largely redundant because the reflectance and fluorescence polarization images can generally provide sufficiently accurate information for the majority of pixels in a tissue image. In contrast, a combination of the RFPI technique with a two-wavelength spectral analysis of a region of an image (e.g., several pixels or more) that appears suspicious to a surgeon who inspects the images intraoperatively can provide a more practical approach that is suitable for use in clinical practice.

Accordingly, analysis of the optical density and corresponding wavelength-dependent derivatives for both cancerous tissue and healthy tissue structures can facilitate more accurate delineation of nonmelanoma skin cancer when performing RFPI procedures. A spectral range corresponding to the absorption bands of the MB dye can be used for reliable differentiation of cancerous tissue, e.g., BCCs. The tumor demarcation technique described herein, e.g., includes imaging at multiple wavelengths. For example, spectroscopic analysis at two (2) interrogation wavelengths, e.g., about 615 nm and about 665 nm, can significantly improve such differentiation because the observed optical density and corresponding wavelength-dependent derivatives of certain normal skin structures may be significantly different from those of cancerous tissue at these wavelengths. Thus, embodiments of the present invention can provide a method and apparatus that combines reflectance and fluorescence polarization imaging with such spectral analysis that facilitates real-time, accurate cancer delineation. Such delineation can be performed, e.g., pre-operatively, intra-operatively, and/or post-operatively, and can also be performed either in vivo or ex vivo.

Wavelengths used for particular tissue analyses may be selected based on various criteria. For example, if blood is present in the sample, use of a wavelength at or near 410 nm that is more strongly absorbed by blood may be used to assess and/or facilitate removal of the effects of blood absorption and/or to distinguish effects of blood and dye in the spectral analysis procedure. A reference wavelength between about 1100 nm and about 1300 nm may also be used, for example, for distinguishing certain types of tissue structures. There may be few strong absorbers in this wavelength range, such that scattering effects may be dominant. In contrast, water absorption is relatively strong around a wavelength of about 1400 nm, so this wavelength region may not be as desirable for examining scattering effects.

The foregoing merely illustrates the principles of the invention. Various modifications and alterations to the described embodiments will be apparent to those skilled in the art in view of the teachings herein. It will thus be appreciated that those skilled in the art will be able to devise numerous techniques which, although not explicitly described herein, embody the principles of the invention and are thus within the spirit and scope of the invention. All patents and publications cited herein are incorporated herein by reference in their entireties.

TABLE 1
Characteristics of cancer-containing tissue samples studied.

| | | | Type | | L × W × H, |
|---|---|---|---|---|---|
| # | Age | Gender | of lesion | Site | cm |
| 1 | 84 | M | nodular BCC | right temple | 1.8 × 0.8 × 0.25 |
| 2 | 64 | M | nodular BCC | left temple | 1.5 × 0.7 × 0.21 |
| 3 | 73 | M | nodular BCC | left neck | 1.4 × 0.7 × 0.21 |
| 4 | 75 | M | nodular BCC | left helix | 2.1 × 0.8 × 0.25 |
| 5 | 74 | F | nodular BCC | left nasal tip | 2.8 × 1.0 × 0.27 |
| 6 | 64 | F | micronodular | right middle | 1.0 × 0.4 × 0.18 |
| | | | BCC | eyebrow | |
| 7 | 58 | F | nodular BCC | middle nasal | 1.3 × 0.6 × 0.20 |
| | | | | bridge | |
| 8 | 87 | F | nodular BCC | left nose | 1.9 × 0.9 × 0.23 |
| 9 | 67 | M | nodular BCC | left nasal tip | 1.3 × 0.9 × 0.21 |
| 10 | 59 | M | superficial | left scalp | 2.0 × 1.7 × 0.24 |
| | | | and nodular | | |
| | | | BCC | | |
| 11 | 60 | M | nodular BCC | left upper | 0.7 × 0.3 × 0.12 |
| | | | | forehead | |
| 12 | 48 | M | nodular BCC | right nasal wing | 0.9 × 0.3 × 0.12 |
| 13 | 83 | M | superficial | left temple | 2.4 × 1.0 × 0.23 |
| | | | and nodular | | |
| | | | BCC | | |
| 14 | 58 | M | nodular BCC | left nasal sidewall | 1.0 × 0.5 × 0.19 |
| 15 | 52 | F | nodular BCC | right upper lip | 1.7 × 0.7 × 0.20 |
| 16 | 76 | F | nodular BCC | left middle nasal | 1.5 × 0.8 × 0.20 |
| | | | | bridge | |
| 17 | 70 | F | nodular BCC | left temple | 2.5 × 1.1 × 0.27 |
| 18 | 64 | M | nodular BCC | left upper neck | 2.2 × 1.2 × 0.25 |
| 19 | 66 | M | nodular BCC | right nasal bridge | 1.7 × 0.8 × 0.22 |
| 20 | 78 | M | nodular BCC | middle scalp | 1.4 × 0.6 × 0.21 |

TABLE 2
Spectral regions of maximal optical contrast between cancer
and normal skin structures as determined by unpaired two-tailed
t-test at a significance level of 0.05.

| | | | derivatives | |
|---|---|---|---|---|
| | | | Wave- | |
| | optical density | | length | |
| | Wavelength | | range of | |
| Healthy | range of | | statis- | |
| structure | statistically | | tically | |
| compared | significant | | significant | |
| with | difference, | | difference, | |
| tumor | nm | p-values | nm | p-values |
| collagen | [595; 725] | 0 ≦ p ≦ | 595 | p = 0.0114 |
| | | 0.00045 | 625 | p = 0.0095 |
| | | | 635 | p = 0.0021 |
| | | | [675; 725] | 0 ≦ p ≦ 0.036 |
| epidermis | [615; 715] | 0 ≦ p ≦ 0.019 | [595; 665] | 0 ≦ p ≦ 0.0017 |
| | | | [685; 725] | 0 ≦ p ≦ |
| | | | | 0.000001 |
| sub- | [595; 725] | 0 ≦ p ≦ | 595 | p = 0.000031 |
| cutaneous | | 0.000001 | [675; 725] | 0 ≦ p ≦ 0.017 |
| fat | | | | |
| hair | — | — | 615 | p = 0.035 |
| follicle | | | 705 | p = 0.021 |
| | | | 725 | p = 0.009 |
| sebaceous | [625; 695] | 0.001 ≦ p ≦ | 645 | p = 0.034 |
| gland | | 0.032 | [685; 705] | 0.0008 ≦ p ≦ |
| | | | | 0.035 |
| | | | 725 | p = 0.011 |

1. An apparatus for imaging a tissue region, comprising:
a light emitter operable to emit a first light having a first wavelength and a second light having a second wavelength onto the tissue region;
a light detector operable to detect a third light remitted from the tissue region based on the first light and a fourth light remitted from the tissue region based on the second light, and to generate signals based on the third and fourth lights; and
an analyzer operable to image at least one tissue structure within said tissue region based on the signals provided by said light detector.

2. The apparatus according to claim 1, wherein said third light and said fourth light are at least partially based on an interaction between a dye provided in the tissue region and said first and second lights.

3. The apparatus according to claim 2, wherein the dye comprises at least one of methylene blue or toluidine blue.

4. The apparatus according to claim 3, wherein the dye comprises methylene blue and at least one of said first wavelength or said second wavelength is between about 600 nm and about 735 nm.

5. The apparatus according to claim 4, wherein at least one of said first wavelength or said second wavelength is about 615 nm or about 665 nm.

6. The apparatus according to claim 2, wherein at least one of said first wavelength or said second wavelength is approximately the same as a wavelength at which there is a local absorption maximum for said dye.

7. The apparatus according to claim 2, wherein at least one of said first light or said second light is polarized, and wherein said light detector is further operable to detect a polarization direction of at least one of said third light or said fourth light at a plurality of particular locations within said tissue region.

8. The apparatus according to claim 7, wherein said analyzer is further operable to generate at least one image of the tissue region based on the polarization directions of said first, second, third, and fourth lights.

9. The apparatus according to claim 8, wherein said at least one image is at least one of a reflectance polarization image or a fluorescence polarization image.

10. The apparatus according to claim 9, wherein said analyzer is further operable to identify said at least one type of tissue structure based on said at least one image.

11. The apparatus according to claim 2, wherein said analyzer further comprises a computer-readable medium, and wherein:
a plurality of wavelength-dependent optical interaction parameters associated with a plurality of tissue structures are provided on said computer-readable medium; and
said analyzer is configured to compare said signals with said parameters to identify said at least one tissue structure within said tissue region.

12. The apparatus according to claim 11, wherein said computer-readable medium comprises at least one of a volatile memory arrangement, a non-volatile memory arrangement, an optical storage medium, a magnetic storage medium, or a medium accessible by said analyzer over a network.

13. The apparatus according to claim 2, wherein the analyzer is further operable to determine at least one of a reflectance or an optical density for said first and second wavelengths at a plurality of particular locations within said tissue region based on said first, second, third, and fourth lights.

14. The apparatus according to claim 13, wherein said analyzer is further operable to generate at least one image of the tissue region based on said reflectance or said optical density.

15. The apparatus according to claim 14, wherein said analyzer is further operable to determine said least one tissue structure based on said at least one image.

16. The apparatus according to claim 2, wherein the analyzer is further operable to determine derivative information for at least one of a reflectance or an optical density with respect to wavelength at a particular wavelength and at least one particular location within said tissue region based on said first, second, third, and fourth lights, and to image the at least one tissue structure based on the derivative information.

17. The apparatus according to claim 16, wherein the derivative information comprises at least one of a first derivative or a second derivative.

18. The apparatus according to claim 17, wherein a difference between the wavelengths of the first and second lights is between about 10 nm and about 30 nm.

19. The apparatus according to claim 17, wherein said analyzer is further operable to:
determine said derivative information at a plurality of locations within the tissue region, and to generate at least one image of the tissue region based on said derivative information; or
identify said least one tissue structure based on said derivative information.

20. (canceled)

21. The apparatus of claim 1, wherein said at least one tissue structure comprises cancerous tissue.

22. The apparatus of claim 1, wherein said apparatus is operable to at least one of image or identify said at least one tissue structure substantially in real time.

23. A method for imaging a tissue region comprising the steps of:
treating said tissue region with a contrast agent;
directing a first light having a first wavelength onto the tissue region;
directing a second light having a second wavelength onto the tissue region;
detecting a third light remitted from the tissue region based on the first light and a fourth light remitted from the tissue region based on the second light; and
generating an image of at least one tissue structure within said tissue region based on said first, second, third, and fourth lights.

24. The method of claim 23, wherein said contrast agent comprises at least one of methylene blue or toluidine blue.

25. The method of claim 24, wherein the contrast agent comprises methylene blue and at least one of said first wavelength or said second wavelength is;
between about 600 nm and about 735 nm; or
about 615 nm or about 665 nm.

26. (canceled)

27. The method of claim 23, wherein:
at least one of said first wavelength or said second wavelength is approximately the same as a wavelength at which there is a local absorption maximum for said contrast agent; or
at least one of said first light or said second light is polarized, and wherein said generating step comprises generating said image based on a polarization direction of at least one of said third light or said fourth light at a plurality of particular locations within said tissue region.

28. (canceled)

29. The method of claim 27, wherein said generating step comprises generating at least one image of the tissue region based on the polarization directions of said first, second, third, and fourth lights.

30. The method of claim 29, wherein said at least one image is at least one of a reflectance polarization image or a fluorescence polarization image.

31-36. (canceled)

37. The method of claim 23, wherein said at least one tissue structure comprises cancerous tissue.

38. (canceled)
